(19) United States
(12) Patent Application Publication
(10) Pub. No.: US 2018/0132936 A1
(43) Pub. Date: May 17, 2018

(54) AN OPTICAL SYSTEM

(71) Applicant: KONINKLIJKE PHILIPS N.V., EINDHOVEN (NL)

(72) Inventor: KIRAN KUMAR THUMMA, EINDHOVEN (NL)

(21) Appl. No.: 15/572,352

(22) PCT Filed: May 30, 2016
(86) PCT No.: PCT/EP2016/062123

§ 371 Date: Nov. 7, 2017

(30) Foreign Application Priority Data
May 28, 2015 (EP) 15169662.2

(51) Int. Cl. A61B 18/20

(52) U.S. Cl. CPC A61B 18/203; A61B 2017/00066

(57) ABSTRACT

The present application relates to an optical system (1) comprising a light beam generator (10) configured to generate a light beam (2) and direct it along an optical path (3), an optical element (19, 24) in said optical path (3) and on which said light beam (2) is incident for redirecting said light beam (2), a sensor (4, 5) attached to said optical element (19, 24), the sensor (4, 5) being configured to generate information indicative of a characteristic of said light beam (2) that is incident on said optical element (19, 24), and a controller (6) configured to adjust one or more characteristics of said optical system (1) in dependence on the information generated by said sensor (4, 5).

### FIELD OF THE INVENTION

The present invention relates to an optical system. The present invention also relates to a laser shaving device for cutting hair comprising the optical system. The present invention also further relates to a method of adjusting a characteristic of the optical system.

### BACKGROUND OF THE INVENTION

It is known to use a light beam, such as a laser beam, and an optical system to cut hair as an alternative to an arrangement of mechanical blades. The optical system directs the laser beam across a recess in a laser shaving device substantially parallel and proximate to a skin surface. Hair exposed to the laser beam absorbs the energy from the laser beam and is severed by vaporisation or laser induced optical breakdown.

However, a small misalignment in a high magnification optical system can lead to a larger shift in the laser beam position. The shift in laser beam position may mean that there is less optical power available to cut hair. Therefore, the shift in laser beam position may result in hair not being cut. The laser beam may be shifted towards the skin surface which can result in irritation. Alternatively, the laser beam may be shifted away from the skin surface which can result in hair that is too long.

Furthermore, another common problem faced when using laser shaving devices is that the skin surface varies in height. Therefore, the distance between the laser beam and skin surface varies which can also result in either irritation or hair that is too long. It is known to deactivate the laser beam if the distance between the laser beam and skin surface becomes too small in order to avoid causing irritation to the skin surface. However, deactivating the laser beam results in hairs not being cut and a longer shaving ritual.

### SUMMARY OF THE INVENTION

It is an object of the invention to provide an optical system which substantially alleviates or overcomes the problems mentioned above.

According to the present invention, there is provided an optical system comprising a light beam generator configured to generate a light beam and direct it along an optical path, an optical element in said optical path and on which said light beam is incident for redirecting said light beam, a sensor attached to said optical element, the sensor being configured to generate information indicative of a characteristic of said light beam that is incident on said optical element, and a controller configured to adjust one or more characteristics of said optical system in dependence on the information generated by said sensor.

With this arrangement, the optical system can recalibrate itself and/or maintain the correct characteristics. Therefore, time consuming and tedious procedures, such as alignment, do not need to be undertaken to calibrate the optical system.

The optical path may include an operative portion in which said light beam is configured to perform a function, said optical element being configured to redirect said light beam into said operative portion.

With this arrangement, the size of the optical system can be reduced or manipulated and the optical path can be more complex. Furthermore, the operative portion of the light beam can be directed to where it is needed to perform its function.

The optical path may include an operative portion in which said light beam is configured to perform a function, said optical element being configured to redirect said light beam out of said operative portion.

With this arrangement, the size of the optical system can be reduced or manipulated and the optical path can be more complex. Furthermore, the operative portion of the light beam can be directed away to an energy dissipater to ensure the light beam does not damage the optical system or a user.

The optical element may have a substantially reflective surface and the sensor may be configured to generate information indicative of a characteristic of said light beam incident on said substantially reflective surface.

Therefore, a portion of the light beam may not be reflected by the reflective surface. Furthermore, the light that passes through the substantially reflective surface may be measured by the sensor.

The sensor may be attached to a rear face of the substantially reflective surface, the substantially reflective surface being configured to allow a non-reflected portion of the light beam to pass through said reflective surface for detection by the sensor.

With this arrangement, the sensor can generate information indicative of a characteristic of the light beam at the substantially reflective surface and does not interfere with the optical path of the light beam whilst reducing the size of the optical system. Furthermore, the sensor is not damaged by the high intensity light beam incident on the reflective surface.

The optical path may include an operative portion in which said light beam is configured to perform a function, said optical element having a first part configured to redirect said light beam into said operative portion and, a second part configured to redirect said light beam out of said operative portion.

With this arrangement, the optical path of the light beam can be more complex allowing for a more compact packing of the optical system. Therefore, devices which use the optical system can be smaller and easier to use. Furthermore, the length of the operative portion of the light beam can be set to suit the application and ensure that the operative portion of the light beam travels along the correct path.

The first and second parts of said optical element may each have a substantially reflective surface and the sensor may comprise first and second sensor elements configured to generate information indicative of a characteristic of said light beam incident on said substantially reflective surfaces of said first and second parts, respectively.

With this arrangement, the controller receives information about the position of the light beam at both ends of its operative portion. Therefore, the position of the operative portion of the light beam between them is known. This allows the controller to make adjustments to ensure that the optical system is aligned.

The first sensor element may be attached to a rear face of the substantially reflective surface of the first part of the optical element and the second sensor element may be attached to a rear face of the substantially reflective surface of the second part of the optical element, the substantially reflective surfaces of the first and second parts may be configured to allow a non-reflected portion of the light beam to pass through said reflective surfaces for detection by the first and second sensor elements, respectively.

With this arrangement, the controller receives information about the position of the light beam at both ends of its operative portion. Therefore, the position of the operative portion of the light beam is known and can be realigned and maintained. Furthermore, the sensors attached to the rear faces reduce the space required by the optical system and eliminates any error in the optical path from the reflective surface to the sensor. The non-reflected portion of light is of a low intensity that does not damage the sensors and the optical path of the light beam is not interfered with.

The sensor may be configured to detect a position of said light beam.

With this arrangement, the position of the light beam at the beginning or the end of the optical path of the operative portion can be determined and compared to a reference value. Therefore, if the operative portion of the light beam is not in the desired position it can then be automatically corrected.

The optical system may further comprise an actuator operable to automatically adjust a position of the operative portion of said light beam, the controller may be configured to receive a signal from the sensor and to operate the actuator in dependence of said signal if the position of the operative portion of the light beam is outside a predetermined position.

Therefore, the optical system is able to ensure it is constantly aligned correctly whilst use and maintain the correct alignment.

The optical system may further comprise a lens arrangement configured to alter at least one characteristic of said operative portion of said optical path.

With this arrangement, the reflective surface of the optical element can remain stationary or restricted to only translational movement. This means that the reflective surface can be fixed in position which helps to eliminate or at least significantly reduce any error in the position of the optical path of the operative portion of the light beam. By fixing the position of the reflective surface, the optical path of the operative portion of the light beam can be transposed and remain parallel to the original position by manipulation of the lens arrangement rather than extending at an angle to the original position when reflected by a rotated reflective surface which can lead to misalignment and damage to other parts of the optical system or irritation to living tissue.

According to another aspect of the present invention, there is provided a laser shaving device comprising an optical system according to an optical system of the present invention wherein said light beam is configured to cut hairs.

With this arrangement the optical system can be used to shave hair on a user's skin without irritating the skin whilst providing a close shave.

The device may comprise a recess, the optical system being configured so that the operative portion of the light beam extends across the recess.

Therefore, the optical system at least reduces irritation being caused to the skin surface by ensuring the light beam travels across the recess and not directly towards the skin surface.

According to yet another aspect of the present invention, there is provided a method of adjusting a characteristic of an optical system which may have a light beam generator configured to generate a light beam and direct it along an optical path, an optical element in said optical path for redirecting said light beam and on which said light beam is incident, and a sensor attached to said optical element, wherein the method may include operating the optical system to direct the light beam along said optical path so that it is incident on said optical element, generating information indicative of a characteristic of the light beam incident on said optical element using the sensor and adjusting one or more characteristics of said optical system in dependence on the information generated by the sensor.

The method of adjusting a characteristic of the optical system, wherein the optical system may further comprise an actuator and the sensor may be configured to detect a position of the light beam, wherein the step of adjusting one or more characteristics of said optical system may comprise controlling the actuator to automatically adjust a position of an operative portion of said optical path along which said light beam travels towards a predetermined position.

Therefore, the optical system is able to ensure it is constantly aligned correctly whilst in use and maintain the correct alignment.

These and other aspects of the invention will be apparent from and elucidated with reference to the embodiments described hereinafter.

### BRIEF DESCRIPTION OF THE DRAWINGS

Embodiments of the invention will now be described, by way of example only, with reference to the accompanying drawings, in which:

FIG. 1 shows a front view schematic diagram of an optical system;

FIG. 2 shows a schematic block diagram of the optical system of FIG. 1;

FIG. 3 shows a perspective view of a laser shaving device for cutting hair having a recess and comprising the optical system of FIG. 1;

FIG. 4 shows a front view schematic diagram of the recess end of the laser shaving device of FIG. 3;

FIG. 5 shows a front view schematic diagram of the recess end of an alternative embodiment of the laser shaving device; and

FIG. 6 shows a schematic block diagram of the laser shaving device of FIG. 5.

### DETAILED DESCRIPTION OF THE EMBODIMENTS

Referring to FIG. 1, there is shown an optical system 1. The optical system 1 is configured to guide a light beam 2 along an optical path 3. The optical path 3 is the route along which the light beam 2 travels.

The optical system 1 comprises at least one sensor 4, 5. The at least one sensor 4, 5 is configured to generate information indicative of the position of the light beam 2 at at least one stage along the optical path 3.

The optical system 1 further comprises a controller 6. The controller 6 is configured to determine whether the light beam 2 is following the desired optical path 3. The controller 6 is configured to determine a characteristic of the light beam 2 on the optical path 3 based on the information generated by the at least one sensor 4, 5. The characteristic of the light beam 2 may be, for example, but not limited to the position of the light beam 2.

The optical system 1 further comprises an actuator 7. The actuator 7 is configured to adjust the optical system 1 so that the light beam 2 does follow the desired optical path 3. The actuator 7 adjusts components of the optical system 1 based on instructions from the controller 6. Therefore, the optical system 1 may automatically align itself.

The optical system 1 comprises a light beam generator 10. The light beam generator 10 is configured to generate the light beam 2. The light beam 2 may be, for example, but not limited to, collimated light or a laser beam. Therefore, the light beam generator 10 is at the beginning of the optical path 3 of the light beam 2. The light beam generator 10 is configured to direct the light beam 2 towards the rest of the optical system 1. In the present embodiment, shown in FIG. 1, the light beam 2 emitted by the light beam generator 10 is initially directed downwards. The optical system 1 redirects the light beam 2 along the desired optical path 3. The light beam generator 10 may be, for example, but not limited to, a laser diode.

The light beam generator 10 may be configured to generate the light beam 2 with an intensity to match the optical system's application. The optical system 1 may be, for example, but not limited to, used to trim a beard.

In the present embodiment, the optical system 1 further comprises a lens arrangement 11. The lens arrangement 11 is configured to focus and direct the light beam 2 emitted from the light beam generator 10. In the embodiment shown in FIG. 1, the lens arrangement 11 comprises a collimating lens 12. The collimating lens 12 reduces or eliminates divergence of the light beam 2 emitted from the light beam generator 10 towards the rest of the optical system 1. The collimating lens 12 creates a collimated portion 13 of the light beam 2.

The lens arrangement 11 further comprises at least one focus lens 14. The present embodiment comprises two focus lenses 14. The focus lenses 14 are configured to converge the light beam 2 and/or direct the collimated portion 13 of the light beam 2 towards the rest of the optical system 1. The focus lenses 14 create an incident portion 15 of the light beam 2.

The optical system 1 further comprises at least one optical element 19, 24. A first optical element 19 is located proximate to the light beam generator 10 along the optical path 3. The first optical element 19 is distal to the end of the optical path 3 of the light beam 2.

The first optical element 19 comprises a first reflective surface 20. The first reflective surface 20 is configured to redirect the incident portion 15 of the light beam 2 into an operative portion 21 of the light beam 2 towards the end of the optical path 3. The incident portion 15 of the light beam 2 is directed onto the first reflective surface 20 by the lens arrangement 11. In an alternative embodiment comprising a single optical element 19 having a reflective surface 20 the incident portion 15 of the light beam 2 may be the operative portion 21 of the light beam 2 and the optical element 19 may reflect the light beam 2 out of the operative portion 21.

The incident portion 15 of the light beam 2 is reflected as the operative portion 21 of the light beam 2 by the first reflective surface 20. The operative portion 21 of the light beam 2 is, for example, but not limited to, used to treat living tissue or trim a beard.

The at least one sensor of the optical system 1 comprises a first sensor 4. In the present embodiment, the first sensor 4 is a position sensor, although the sensor is not limited thereto. The first sensor 4 is configured to generate information indicative of the position of the beginning of the operative portion 21 of the light beam 2 relative to a reference point. Alternatively, the first sensor 4 may generate information indicative of the position of the end of the incident portion 15 of the light beam 2 which is the same as the position of the beginning of the operative portion 21.

In the present embodiment, the reference point is at the centre of the first sensor 4. However, it will be understood that in an alternative embodiment, the first sensor 4 may be calibrated to have the reference point at a point other than its centre. It will be understood that in an alternative embodiment the sensor may measure another characteristic of the light beam 2. In such an embodiment, it may not be essential to measure the characteristic relative to the reference point.

The first sensor 4 is attached to the first reflective surface 20 of the first optical element 19. The first sensor 4 is attached to a rear face 22 of the first reflective surface 20. The rear face 22 of the first reflective surface 20 is the side which the incident portion 15 of light beam 2 is not directed onto. The first sensor 4 covers the whole of the rear face 22 of the first reflective surface 20. However, it will be understood that in an alternative embodiment, the first sensor 4 may only partially cover the rear face 22 of the first reflective surface 20.

The first reflective surface 20 does not reflect all of the light of the incident portion 15 of the light beam 2. A small amount of light of the light beam 2 passes through the first reflective surface 20 instead of being reflected. The first sensor 4 is configured to detect the low intensity light which is not reflected by the first reflective surface 20 and generate information indicative of a characteristic of the operative portion 21 of the light beam 2, for example, its position.

The optical elements 19, 24 may comprise mirrors or prisms. If the optical element 19,24 is a mirror, the sensor 4 can be attached to its rear surface so that some of the light which is not reflected passes through the mirror and is detected by the sensor 4. Similarly, if the optical element 19,24 is a prism, the sensor 4 may be located on the rear face of the internal surface of the prism on which the light beam is incident. Whilst the majority of the light is reflected off this surface, some of it will pass through the surface to be detected by the sensor 4.

With this arrangement, the position of the light beam 2 at the beginning of the optical path 3 of the operative portion 21 of the light beam 2 can be determined and compared to the reference point. Therefore, the position of the operative portion 21 of the light beam 2 can be monitored and it will be known if the light beam 2 is not in the desired position at that stage of the optical path 3. Furthermore, the first sensor 4 will not be damaged by the high intensity of the light beam 2 and does not interfere with the optical path 3 of the light beam 2.

In the present embodiment, as shown in FIG. 1, the optical system 1 further comprises a second optical element 24. The second optical element 24 is located distal to the light beam generator 10 along the optical path 3 of the light beam 2. Therefore, the first optical element 19 is located between the lens arrangement 11 and the second optical element 24. The second optical element 24 comprises a second reflective surface 25.

The operative portion 21 of the light beam 2 is directed onto the second reflective surface 25 by the first reflective surface 20. The second reflective surface 25 is configured to substantially reflect the light beam 2 out of the operative portion 21. The second reflective surface 25 redirects the light beam 2 away from the first reflective surface 20 of the first optical element 19.

The optical system 1 further comprises a second sensor 5. In the present embodiment, the second sensor 5 is a position sensor, although the sensor is not limited thereto. The second sensor 5 is configured to generate information indicative of the position of the operative portion 21 of the light beam 2 relative to a reference point. In the present embodiment, the reference point is at the centre of the second sensor 5. However, it will be understood that in an alternative embodiment, the sensor may be calibrated to have the reference point at a point other than its centre.

The second sensor 5 is attached to the second reflective surface 25. The second sensor 5 is attached to a rear face 26 of the second reflective surface 25. The rear face 26 of the second reflective surface 25 is the side which the operative portion 21 of light beam 2 is not directed onto. The second sensor 5 covers the whole of the rear face 26 of the second reflective surface 20. However, it will be understood that in an alternative embodiment, the second sensor 5 may only partially cover the rear face 26 of the second reflective surface 25.

With this arrangement, the position of the light beam 2 at the end of the optical path 3 of the operative portion 21 of the light beam 2 can be determined and compared to the reference point. Therefore, the position of the operative portion 21 of the light beam 2 can be monitored and it will be known if the light beam 2 is not in the desired position at that stage of the optical path 3. Furthermore, knowing the position of the operative portion 21 of the light beam 2 at both its beginning and end allows the path of the operative portion 21 of the light beam 2 to be known. Additionally, the second sensor 5 will not be damaged by the high intensity operative portion 21 of the light beam 2 and does not interfere with the optical path 3 of the light beam 2.

With this arrangement, the first and second sensors 4, 5 do not need to be moved. Therefore, the optical path 3 of the light beam 2 can be adjusted by the actuator 7 moving the lens arrangement 11 rather than changing the angle of the first and second reflective surfaces 20, 25 which can lead to misalignment and damage to other parts of the optical system 1 or irritation to living tissue.

In the present embodiment, as shown in FIG. 1, the light beam generator 10 emits the light beam 2 vertically downwards. The incident portion 15 of the light beam 2 is also directed downwards vertically after passing through the lens arrangement 11. The first reflective surface 20 of the first optical element 19 then reflects the incident portion 15 of the light beam 2 through 90 degrees such that the operative portion 21 of the light beam 2 is directed towards the second reflective surface 25 of the second optical element 24. Therefore, the first reflective surface 20 is angled at 45 degrees to the optical path 3 of the incident portion 15 of the light beam 2.

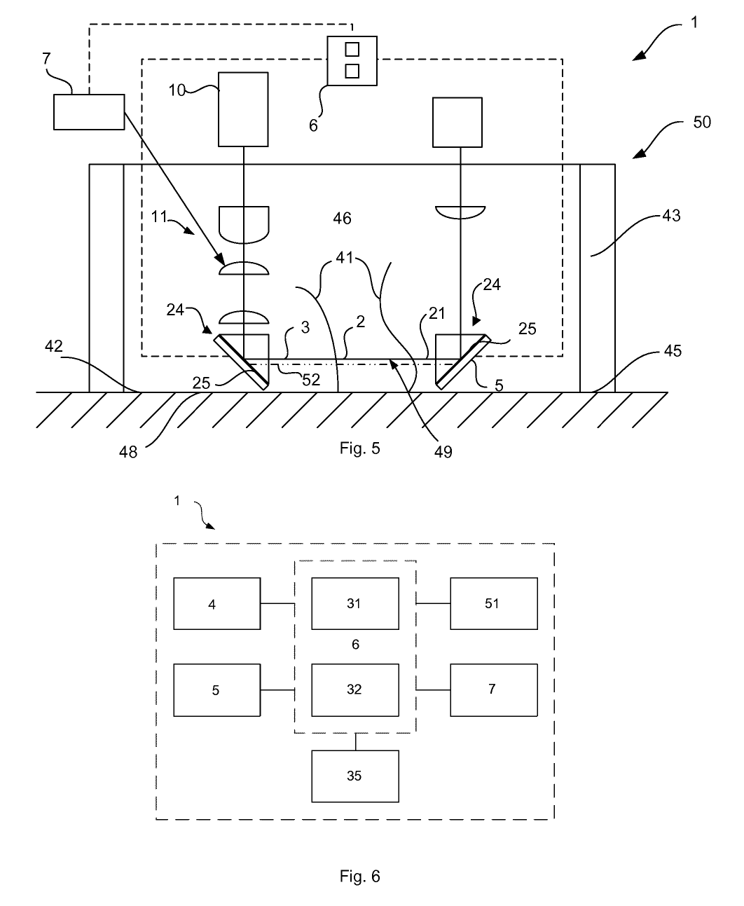

Furthermore, this means that when the optical system 1 is used in a device to, for example, trim a beard, the operative portion 21 of the light beam 2 will extend substantially parallel to a skin surface 42, shown in FIG. 4 and FIG. 5, of a user. This means that the light beam 2 is less likely to cause irritation to skin surface or trim a beard unevenly proximate or distal to the first and second reflective surfaces 20, 25 as it does not extend towards or away from the skin surface 42.

The first reflective surface 20 is spaced from the second reflective surface 25. The first reflective surface 20 is spaced horizontally from the second reflective surface 25. Therefore, the operative portion 21 of the light beam 2 can be used to, for example, but not limited to, treat the skin surface 42 or trim a beard. The distance between the first reflective surface 20 and the second reflective surface 25 is determined by the application of the optical system 1.

With this arrangement, the optical path 3 of the light beam 2 can be more complex allowing for a more compact packing of the optical system 1. Therefore, any device which uses the optical system 1 can be smaller and easier to use. Furthermore, the length of the operative portion 21 of the light beam 2 can be set to suit the application and ensure that the operative portion 21 of the light beam 2 travels along the correct optical path 3.

Furthermore, the first reflective surface 20 extends in a direction that is perpendicular to the second reflective surface 25. Therefore, the second reflective surface 25 is angled at 45 degrees to the operative portion 21 of the light beam 2. The second reflective surface 25 is configured to substantially reflect the operative portion 21 of the light beam 2 through 90 degrees such that a “used” portion 27 of the light beam 2 travels in a direction parallel to, opposite to and spaced from the collimated and incident portions 13, 15 of the light beam 2.

The first reflective surface 20 is in alignment with the second reflective surface 25. That is, the first reflective surface 20 is not vertically spaced from the second reflective surface 25. Therefore, when the incident portion 15 of the light beam 2 has an incident angle of 45 degrees and is positioned in the centre of the first sensor 4, the operative portion 21 of the light beam 2 is positioned in the centre of the second sensor 5. Furthermore, any movement in the position of the incident portion 15 of the light beam 2 on the first sensor 4 will be copied by the operative portion 21 of the light beam 2 on the second sensor 5, so long as the incident beam is at 45 degrees to the second reflective surface 25.

Therefore, the reference points on the first and second sensors 4, 5 can be aligned such that the first and second sensors 4, 5 generate the same information when the light beam 2 travels along the correct optical path 3.

The optical system 1 further comprises an energy dissipater 28. The energy dissipater 28 is configured to convert or absorb the remaining light energy of the light beam 2 so that it is no longer a hazard. The energy dissipater 28 is located at the end of the optical path 3. Therefore, the light beam 2 will not cause damage to any of the components of the optical system 1. The optical system 1 further comprises a dissipating lens 29. The dissipating lens 29 is configured to reduce the intensity of the “used” portion 27 of the light beam 2 before it reaches the energy dissipater 28.

However, it will be appreciated that the first and second reflective surfaces 20, 25 of the optical elements 19, 24 may be orientated differently or have different reflective angles depending on the position and orientation of the other parts of the optical system 1, such as the light beam generator 10, the lens arrangement 11, and the energy dissipater 28.

The at least one reflective surface 20, 25 may be a mirror or a total internal reflection surface of a prism or any other optically reflective surface. Furthermore, the invention is not limited to two reflective elements.

The at least one sensor 4, 5 may be made from a material which can detect low intensity light. The at least one sensor 4, 5 may be, for example, but not limited to, a beam position sensor or a quadrant sector. The at least one sensor 4, 5 may be able to determine the incident beam displacement relative to the calibrated sensor. The at least one sensor 4, 5 may be a line sensor or an array of multiple point sensors.

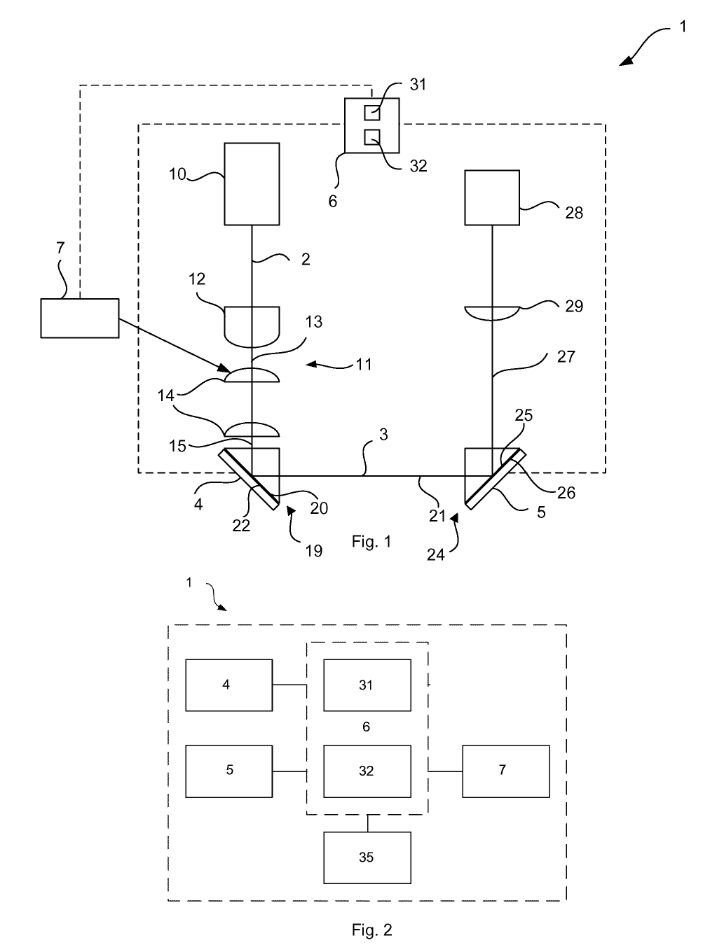

The optical system 1 further comprises the controller 6, shown in FIG. 1 and FIG. 2. The controller 6 is configured to determine the error in the light beam 2 position in dependence on the information generated by the at least one sensor 4, 5 and command the actuator 7 to adjust the optical system 1 to correct the error. The controller 6 comprises a processor 31. The controller 6 further comprises a memory 32.

The processor 31 may take any suitable form. For instance, the processor 31 may be or include a microcontroller, plural microcontrollers, circuitry, a single processor, or plural processors. The processor 31 may be formed of one or multiple modules.

The memory 32 may take any suitable form. The memory 32 may include a non-volatile memory and/or RAM. The non-volatile memory may include read only memory (ROM), a hard disk drive (HDD) or a solid state drive (SSD). The memory 32 stores, amongst other things, an operating system. The memory 32 may be disposed remotely. The RAM is used by the processor 31 for the temporary storage of data.

The operating system may contain code which, when executed by the controller 6, controls the operation of the hardware components in the optical system 1. The controller 6 may be able to cause one or more objects, such as one or more profiles, to be stored remotely or locally by the memory 32. The controller 6 may be able to refer to one or more objects, such as one or more profiles, stored by the non-volatile memory and upload the one or more stored objects to the RAM.

The operation of the optical system 1 will now be described with reference to FIG. 1 and FIG 2. The optical system 1 is activated by a user by the use of a user input 35, discussed hereinafter. The user input 35 comprises a user interface. Optionally, the optical system 1 includes controls and/or displays for adjusting an operating characteristic of the optical system 1, such as, for example, but not limited to, the position or power of the light beam 2. The user input 35 allows a user to operate the optical system 1, for example to turn the optical system 1 on and off. The user input 35 may, for example, be a button, touch screen or switch.

The controller 6 commands the light beam generator 10 to generate the light beam 2. The light beam 2 travels through the lens arrangement 11 which directs an incident beam 15 onto the first reflective surface 20 of the first optical element 19.

The first sensor 4 on the rear face 22 of the first reflective surface 20 generates information indicative of the position of the incident portion 15 of the light beam 2 relative to the reference point its centre. The first sensor 4 generates information indicative of the position of the incident portion 15 of the light beam 2 by measuring the low intensity light that is not reflected by the first reflective surface 20. The first sensor 4 sends the information to the controller 6.

The controller 6 compares the information generated by the first sensor 4 indicative of the position of the incident portion 15 of the light beam 2 to the reference point. If the position of the incident portion 15 of the light beam 2 is not the same as the reference point the controller 6 instructs the actuator 7 to act on a component of the optical system 1, in the present embodiment the focus lenses 14, to align the incident portion 15 of the light beam 2 with the reference point on the first sensor 4.

The light beam 2 is reflected by the first reflective surface 20 and the operative portion 21 of the light beam 2 travels to the second reflective surface 25. The second sensor 5 on the rear face 26 of the second reflective surface 25 generates information indicative of the position of the operative portion 21 of the light beam 2 relative to the reference point its centre. The second sensor 5 generates information indicative of the position of the operative portion 21 of the light beam 2 by measuring the low intensity light that is not reflected by the second reflective surface 25. The second sensor 5 sends the information to the controller 6.

The controller 6 compares the information generated by the second sensor 5 indicative of the position of the operative portion 21 of the light beam 2 to the reference point. If the position of the operative portion 21 of the light beam 2 is not the same as the reference point the controller 6 instructs the actuator 7 to act on a component of the optical system 1, in the present embodiment the focus lenses 14, to align the operative portion 21 of the light beam 2 with the reference point on the second sensor 5.

The feedback loop continues throughout the time that the light beam 2 is activated. Therefore, once the optical system 1 has correctly aligned itself, it continues to monitor the position of the light beam 2 at various points along the optical path 3 to ensure that the system does not fall out of alignment. An advantage of the feedback loop in the optical system 1 is that the optical system 1 is able to automatically align itself with the reference point or a predetermined distance from the reference point.

In the present embodiment, if either the incident portion 15 or operative portion 21 of the light beam 2 are positioned at the reference point but the other is not, then the actuator 7 must realign the focus lenses 14 so that the incident portion 15 of the light beam 2 meets the reference point at a 45 degree angle to the first reflective surface 20.

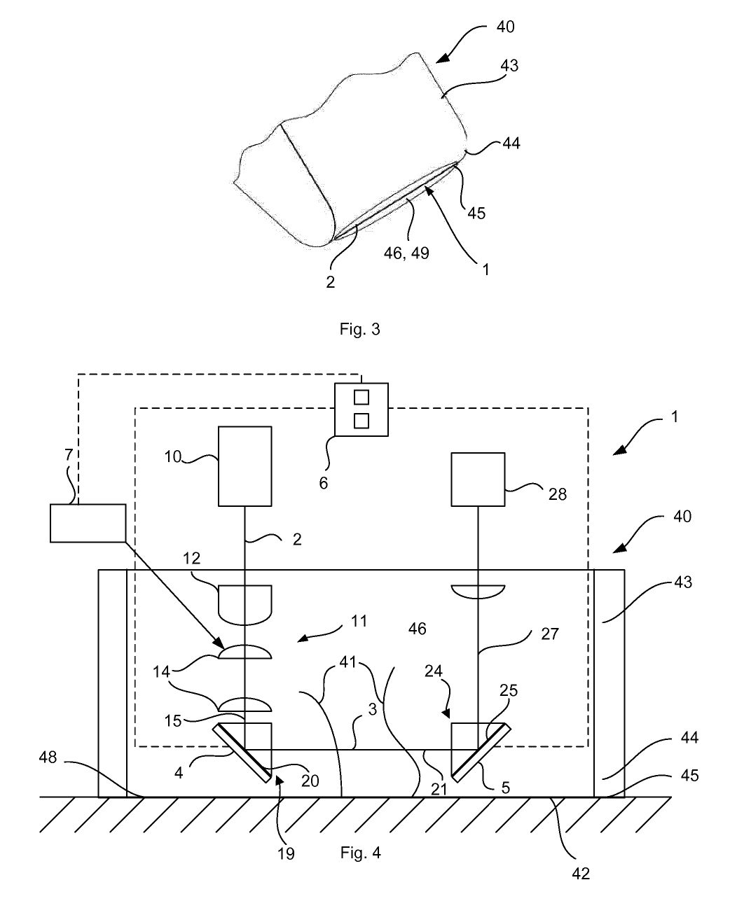

As shown in FIG. 3 and FIG. 4, a laser shaving device 40 comprises the optical system 1. The laser shaving device 40 is configured to cut hair 41 extending from the skin surface 42.

The laser shaving device 40 comprises a housing 43. The housing 43 may comprise a guard 44. The guard 44 may be a hair and skin manipulation module. The housing 43 has a skin engaging face 45. The skin engaging face 45 is configured to be placed against the skin surface 42. The skin surface 42 may be, for example, but not limited to, the face or leg of a user or a person being treated.

The skin engaging face 45 comprises a recess 46. In the present embodiment, the centre of the recess 46 is concentric with the centre of the skin engaging face 45. The recess 46 has a substantially oval cross-section. However, it will be understood that the shape of the cross-section is not limited thereto. For example, the cross-section of the recess 46 may be rectangular.

The recess 46 is a slit. The recess 46 may be greater than or equal to 0.3 mm and less than or equal to 10 mm wide in the direction of the shaving stroke, depending on the application of the laser shaving device 40: shaver or trimmer. The recess 46 in a shaver may be greater than or equal to 0.3 mm and less than or equal to 2 mm wide in the direction of the shaving stroke. The recess 46 in a trimmer may be greater than or equal to 2 mm and less than or equal to 10 mm wide in the direction of the shaving stroke. The recess width helps to control the doming of the skin surface 42. In the present embodiment, the width of the recess 6 is 0.8 mm.

In the present embodiment, the skin engaging face 45 lies in a plane 48 that extends across the recess 46. The plane 48 extends generally perpendicular to the longitudinal axis of the housing 43 of the laser shaving device 40. However, it will be understood that for ergonomic reasons the angle between the plane 48 and the longitudinal axis of the housing 43 may vary.

The optical system 1 is located within the housing 43 of the laser shaving device 40. The optical system 1 is located at least partially within the recess 46. The recess 46 comprises a cutting zone 49. When the skin engaging face 45 of the laser shaving device 40 is placed against the skin surface 42 and moved along it, the skin surface 42 and any hair 41 on the skin surface 42 may extend into the cutting zone 49. The operative portion 21 of the light beam 2 is used to cut hairs 41 which extend into the cutting zone 49 by evaporation due to optical absorption or light induced optical breakdown. The operative portion 21 of the light beam 2 is the cutting section.

The optical system 1 directs the operative portion 21 of the light beam 2 across the recess 46 so that it is parallel to and spaced from the plane 48 of the recess 46 on which the skin engaging face 45 lies. The housing 42, or more specifically, the guard 44, mechanically spaces the operative portion 21 of the light beam 2 from the skin surface 42. The optical system 1 directs the operative portion 21 of the light beam 2 such that it extends across the recess 46 proximate to the plane 48. The point at which the operative portion 21 of the light beam 2 travels across the recess 46 is the cutting zone 49.

In this way, when the skin engaging face 45 of the housing 42 is placed against the skin surface 42, the operative portion 21 of the light beam 2 is substantially parallel to and spaced from the skin surface 42.

In the present embodiment, the light beam 2 emitted by the light beam generator 10 is initially directed downwards towards the skin surface 42. The optical system 1 redirects the incident portion 15 of the light beam 2 along the desired optical path 3.

The first optical element 19 is positioned on one side of the cutting zone 49. The first optical element 19 may be fixedly located. The first reflective surface 20 of the first optical element 19 substantially reflects the incident portion 15 of the light beam 2 across the cutting zone 49 of the recess 46 as the operative portion 21 to follow the optical path 3. The first reflective surface 20 substantially reflects the incident portion 15 of the light beam 2 substantially parallel to, and spaced from, the plane 48 that extends across the recess 46 of the laser shaving device 40. The first reflective surface 20 is at a 45 degree angle to the incident portion 15 of the light beam 2. The first reflective surface 20 reflects the incident portion 15 of the light beam 2 through 90 degrees.

The second optical element 24 is positioned on an opposite side of the cutting zone 49 to the first optical element 19. The second optical element 24 may be fixedly located. The second reflective surface 25 of the second optical element 24 substantially reflects the operative portion 21 of the light beam 2 away from the cutting zone 49 of the recess 46 as the ‘used’ portion 27 of the light beam 2. The ‘used’ portion 27 of the light beam 2 is directed towards the energy dissipater 28 so that it does not interact with the skin surface 42 or another part of the laser shaving device 40. The second reflective surface 25 is at a 45 degree angle to the operative portion 21 of the light beam 2. The second reflective surface 25 reflects the operative portion 21 of the light beam 2 through 90 degrees.

By ensuring the incident portion 15 travels vertically and having the first reflective surface 20 at a 45 degree angle to the vertical incident portion 15 of the light beam 2, it is ensured that the operative portion 21 of the light beam 2 travels parallel to the plane 48 that extends across the recess 46. Therefore, the operative portion 21 of the light beam 2 is at a substantially constant height above the skin surface 42. This means that there is less chance that the operative portion 21 of the light beam 2 is directed towards the skin surface 42 which would cause irritation or away from the skin surface 42 which would result in hair on one side of the recess 46 being much longer than on the other.

Furthermore, the first and second reflective surface 20, 25 are horizontally, but not vertically, spaced. Therefore, they are substantially symmetrical about an equidistant point. The result is that the first and second sensors 4, 5 are also substantially symmetrically aligned. Therefore, the reference points on the first and second sensors 4, 5 are at the same vertical distance from the plane 48 that extends across the recess 46. It means that if the information generated by each sensor 4, 5 is not identical then the controller 6 will automatically know that the optical system 1 is not properly aligned and command the actuator 7 to adjust the optical system 1. It will be appreciated that if identical sensors 4, 5 are used then the information generated in the x-direction may be sensed at an equal and opposite distance on each sensor 4, 5 from the reference point.

However, it will be appreciated that the first and second reflective surfaces 20, 25 may not be located at a side of the cutting zone 49 of FIG. 3 and FIG. 4. They may alternatively be located anywhere within the recess 46, depending on the position, orientation and configuration of the other components of the optical system 1. The first and second reflective surfaces 20, 25 may be omitted.

However, the operative portion 21 of the light beam 2 should remain substantially parallel to the plane 48 extending across the recess 46 on which the skin engaging face 45 lies so that the distance between the skin engaging face 45 and the operative portion 21 of the light beam 2 is substantially constant across the cutting zone 49 in the recess 46. Therefore, the operative portion 21 of the light beam 2 will be prevented from irritating the skin surface 42 on one side of the cutting zone 49 and not cutting hair 41 short enough on the other.

The lens arrangement 11 is configured to focus the light beam 2 emitted from the light beam generator 10 using the collimating lens 12 and the focus lenses 14. Other components that the laser shaving device 40 may comprise, which are not shown, may include other optical components such as a filter or windows to limit the passage of detritus in the laser shaving device 40.

Other components necessary for the operation of the laser shaving device 40 may also be located within the housing 42, such as a battery or a connection to an external power cable (not shown). Moreover, the housing 43 of the laser shaving device 40 may also comprise a handle and any switches, buttons or other controls and displays necessary to operate the laser shaving device 40, which may form the user input 35, shown in FIG. 6.

In the present embodiment, the user may use the user input 35 to select a desired stubble length. The desired stubble length corresponds to a point on the first and second sensors 4, 5. Therefore, the controller 6 commands the actuator 7 to adjust the focus lenses 14 to adjust the point at which the light beam 2 contacts the first and second reflective surfaces 20, 25 to match the desired stubble length and then maintain the optical system 1 in that state. The operative portion 21 of the light beam 2 is transposed towards or away from and kept substantially parallel to the skin surface 42.

Referring now to FIG. 5 and FIG. 6, there is shown a front view schematic diagram of the recess end of an alternative embodiment of a laser shaving device 50 and a schematic block diagram of the laser shaving device 50, respectively. The laser shaving device 50 shown in FIG. 5 is generally the same as the embodiment of the laser shaving device 40 described above and so a detailed description will be omitted herein. Furthermore, features and components of the laser shaving device 50 will retain that same terminology and reference numerals. However, the laser shaving device 50 shown in FIG. 5 comprises the optical system 1 and a skin sensor arrangement 51.

In the present embodiment, the skin sensor arrangement 51 comprises a low power light beam 52. The low power light beam 52 has a lower intensity than the operative portion 21 of the light beam 2. The low power light beam 52 is safe to contact the skin surface 42. The low power light beam 52 is emitted by a low power light beam generator (not shown). The low power light beam 52 is a skin distance sensor light beam.

However, it will be understood that in an alternative embodiment, the low power light beam 52 may be a part of the operative portion 21 of the light beam 2. That is, the low power light beam 52 may comprise the part of the operative portion 21 of the light beam 2 whose intensity is safe to contact the skin surface 42. This is possible because the intensity of a light beam 2 decreases as the radius from its centre line increases.

In the present embodiment, the low power light beam 52 follows an optical path which is substantially parallel to the optical path 3 of the light beam 2. Therefore, the low power light beam 52 may pass through the lens arrangement 11 and be reflected by the first reflective surface 20 of the first optical element 19 towards the second reflective surface 25 of the second optical element 24.

The low power light beam 52 extends across the cutting zone 49 of the recess 46 in the housing 43. The low power light beam 52 extends parallel to the plane 48 that extends across the recess 46. The low power light beam 52 is located between the operative portion 21 of the light beam 2 and the plane 48 that extends across the recess 46. Therefore, the position of the skin surface 42 can be determined before it gets too close to the operative portion 21 of the light beam 2.

In an embodiment in which the low power light beam 52 is reflected by the first reflective surface 20, the 45 degree angle ensures that the vertical incident portion (not shown) of the low power light beam is always reflected so that it travels parallel to the plane 48 that extends across the recess 46. Therefore, the true height of the skin surface 42 inside the recess 46 can always be determined so that the skin surface 42 is never too close to the operative portion 21 of the light beam 2 to be damaged.

The low power light beam 52 is reflected by the second reflective surface 25. Some of the light of the low power light beam 52 will be detected by the second sensor 5. The second sensor 5 generates information indicative of the position of the low power light beam 52. The controller 6 is configured to allow the skin surface 42 to obstruct or reflect a certain amount of the low power light beam 52. The amount of the low power light beam 52 that the skin surface 42 can obstruct corresponds to a safety distance.

The controller 6 monitors the information generated by the second sensor 5 indicative of the position of the low power light beam 52. When the controller 6 determines the skin surface 42 has blocked too great a portion of the low power light beam 52, the skin surface 42 is too close to the operative portion 21 of the light beam 2 and may cause irritation. Therefore, the controller 6 commands the actuator 7 to adjust the lens arrangement 11 to move the optical path 3 of the operative portion 21 of the light beam 2 away from the plane 48 that extends across the recess 46.

When the controller 6 determines the skin surface 42 has blocked too small a portion of the low power light beam 52, the skin surface 42 is too far away from the operative portion 21 of the light beam 2 to give the desired length stubble. Therefore, the controller 6 commands the actuator 7 to adjust the lens arrangement 11 to move the optical path 3 of the operative portion 21 of the light beam 2 towards the plane 48 that extends across the recess 46.

However, it will be understood that in an alternative embodiment the skin sensor arrangement 51 may comprise a skin surface sensor (not shown). The skin surface sensor may be, for example, but not limited to, an electronic sensor which is configured to generate information indicative of the distance between the skin surface 42 in the recess 46 and the plane 48 that extends across the recess 46 when the laser shaving device 50 is positioned against the skin surface 42, or an optical sensor, such as a confocal lens which uses optical measuring techniques and does not need to contact the skin surface 42 to measure the distance between the skin surface 42 and the plane 48 that extends across the recess 46 of the laser shaving device 50. The skin surface sensor may be configured to generate information indicative of the position of the skin surface 42 by, for example, triangulation measurement, scattered light measurement and/or shadow measurement.

It will be appreciated that the term “comprising” does not exclude other elements or steps and that the indefinite article “a” or “an” does not exclude a plurality. A single processor may fulfil the functions of several items recited in the claims. The mere fact that certain measures are recited in mutually different dependent claims does not indicate that a combination of these measures cannot be used to an advantage. Any reference signs in the claims should not be construed as limiting the scope of the claims.

Although claims have been formulated in this application to particular combinations of features, it should be understood that the scope of the disclosure of the present invention also includes any novel features or any novel combinations of features disclosed herein either explicitly or implicitly or any generalisation thereof, whether or not it relates to the same invention as presently claimed in any claim and whether or not it mitigates any or all of the same technical problems as does the parent invention. The applicants hereby give notice that new claims may be formulated to such features and/or combinations of features during the prosecution of the present application or of any further application derived therefrom.

1. An optical system comprising:
a light beam generator configured to generate a light beam and direct it along an optical path;
an optical element in said optical path and on which said light beam is incident for redirecting said light beam;
a sensor attached to said optical element, the sensor being configured to generate information indicative of a characteristic of said light beam that is incident on said optical element; and
a controller configured to adjust one or more characteristics of said optical system in dependence on the information generated by said sensor,
wherein the optical element has a substantially reflective surface and the sensor is configured to generate information indicative of a characteristic of said light beam incident on said substantially reflective surface; and
wherein the sensor is attached to a rear face of the substantially reflective surface, the substantially reflective surface being configured to allow a non-reflected portion of the light beam to pass through said reflective surface for detection by the sensor.

2. The optical system according to claim 1, wherein said optical path includes an operative portion in which said light beam is configured to perform a function, said optical element being configured to redirect said light beam into said operative portion.

3. The optical system according to claim 1, wherein said optical path includes an operative portion in which said light beam is configured to perform a function, said optical element being configured to redirect said light beam out of said operative portion.

4. (canceled)

5. (canceled)

6. The optical system according to claim 1, wherein said optical path includes an operative portion in which said light beam is configured to perform a function, said optical element having a first part configured to redirect said light beam into said operative portion and, a second part configured to redirect said light beam out of said operative portion.

7. The optical system according to claim 14, wherein the first and second parts of said optical element each have a substantially reflective surface and the sensor comprises first and second sensor elements configured to generate information indicative of a characteristic of said light beam incident on said substantially reflective surfaces of said first and second parts respectively.

8. The optical system according to claim 7, wherein the first sensor element is attached to a rear face of the substantially reflective surface of the first part of the optical element and the second sensor element is attached to a rear face of the substantially reflective surface of the second part of the optical element, the substantially reflective surfaces of the first and second parts being configured to allow a non-reflected portion of the light beam to pass through said reflective surfaces for detection by the first and second sensor elements, respectively.
9. The optical system according to claim 1, wherein said sensor is configured to detect a position of said light beam.

10. The optical system according to claim 9, comprising an actuator operable to automatically adjust a position of the operative portion of the light beam, the controller being configured to receive a signal from the sensor and to operate the actuator in dependence on said signal if the position of the operative portion of the light beam is outside a predetermined position.

11. The optical system according to claim 1, further comprising a lens arrangement configured to alter at least one characteristic of said operative portion of said optical path.

12. A laser shaving device comprising an optical system according to claim 1, wherein said light beam is configured to cut hairs.

13. The device according to claim 12, comprising a recess, the optical system being configured so that the operative portion of the light beam extends across the recess.

14. A method of adjusting a characteristic of an optical system, said optical system having a light beam generator configured to generate a light beam and direct it along an optical path, an optical element in said optical path for redirecting said light beam and on which said light beam is incident, and a sensor attached to said optical element, the optical element having a substantially reflective surface and the sensor being configured to generate information indicative of a characteristic of said light beam incident on said substantially reflective surface, and wherein the sensor is attached to a rear face of the substantially reflective surface, the substantially reflective surface being configured to allow a non-reflected portion of the light beam to pass through said reflective surface for detection by the sensor,
wherein the method includes operating the optical system to direct the light beam along said optical path so that it is incident on said optical element, generating information indicative of a characteristic of the light beam incident on said optical element using the sensor, and adjusting one or more characteristics of said optical system in dependence on the information generated by the sensor.

15. The method according to claim 14, wherein the optical system comprises an actuator and the sensor is configured to detect a position of the light beam, wherein the step of adjusting one or more characteristics of said optical system comprises controlling the actuator to automatically adjust a position of an operative portion of said optical path along which said light beam travels towards a predetermined position.

16. The device according to claim 11, the device further comprising a housing, the housing comprising a skin engaging face, the skin engaging face being configured to be placed against a skin surface during use, the skin engaging face being configured to lie in a plane which extends across the recess,
wherein the optical system further comprises a low power light beam, the low power light beam having a lower intensity than the operative portion of the light beam such that the low power light beam is safe to contact a skin surface,
wherein the low power light beam extends across the recess parallel to the plane,
wherein the low power light beam is located between the operative portion of the light beam and the plane.
